(19) United States
(12) Patent Application Publication
(10) Pub. No.: US 2012/0003243 A1
(43) Pub. Date: Jan. 5, 2012

(54) YEAST ECTOPICALLY EXPRESSING ABNORMALLY PROCESSED PROTEINS AND USES THEREFOR

(75) Inventors: Susan L. Lindquist, Chestnut Hill, MA (US); Tiago Outeiro, Lisbon (PT)

(73) Assignee: WHITEHEAD INSTITUTE FOR BIOMEDICAL RESEARCH, Cambridge, MA (US)

(21) Appl. No.: 12/781,591

(22) Filed: May 17, 2010

Related U.S. Application Data
(63) Continuation of application No. 10/826,157, filed on Apr. 16, 2004, now abandoned.
(60) Provisional application No. 60/472,317, filed on May 20, 2003.
(60) Provisional application No. 60/463,284, filed on Apr. 16, 2003.

(51) Int. Cl. A61K 39/395; C12Q 1/44; C12Q 1/68; C12Q 1/02; A61P 43/00; A61K 31/7088; A61K 38/02; A61P 25/16; A61K 31/7105; C12N 1/19; C12Q 1/48

(52) U.S. Cl. 424/172.1; 435/254.2; 435/254.21; 435/254.22; 435/254.23; 435/19; 435/6.1; 435/6.13; 435/29; 435/15; 514/44 R; 514/1.1; 514/17.7; 514/44 A

(57) ABSTRACT

Disclosed are yeast ectopically expressing abnormally processed proteins and methods of screening to identify compounds that modulate the function of such abnormally processed proteins in yeast. Compounds identified by such screens can be used to treat or prevent diseases associated with abnormally processed proteins or protein misfolding. Such diseases include Parkinson's Disease, Parkinson's Disease with accompanying dementia, Lewy body dementia, Alzheimer's disease with Parkinsonism, and multiple system atrophy.

### CROSS REFERENCE TO RELATED APPLICATIONS

This application claims priority from U.S. Provisional Application No. 60/463,284, filed Apr. 16, 2003, and U.S. Provisional Application No. 60/472,317, filed May 20, 2003. The entire content of each of these prior applications is incorporated herein by reference in its entirety.

### STATEMENT AS TO FEDERALLY SPONSORED RESEARCH

This invention was made with Government support under grant number NS044829-01 awarded by the National Institutes of Health/National Institute for Neurological Disorders and Stroke. The Government may have certain rights in the invention.

### FIELD OF THE INVENTION

This invention relates to yeast ectopically expressing abnormally processed proteins and screening methods to identify compounds that modulate the function of such abnormally processed proteins.

### BACKGROUND OF THE INVENTION

Alpha-synuclein is a protein that can aggregate and precipitate into dense intracytoplasmic inclusions called Lewy bodies. Lewy bodies are involved in the etiology of a variety of neurologic disorders, including Parkinson's Disease, Parkinson's Disease with accompanying dementia, Lewy body dementia, Alzheimer's disease with Parkinsonism, and multiple system atrophy.

Parkinson's Disease has a prevalence of about 2% after age 65, and, thus, is one of the most common neurodegenerative human disorders. Its pathological hallmarks are: (a) the presence of Lewy bodies (Spillantini M G, et al., 1997. Nature 388:839-40), round cytoplasmic inclusions ˜5-25 μm in diameter, mainly reactive for alpha-synuclein but also for ubiquitin and other proteins; and (b) massive loss of dopaminergic neurons in the pars compacta of the substantia nigra (Fearnley J M, et al., 1991. Brain 114:2283-2301).

Effective treatments for neurodegenerative diseases such as Parkinson's Disease are needed.

### SUMMARY OF THE INVENTION

The invention is based, at least in part, on the discovery that yeast that ectopically express an abnormally processed protein, such as alpha synuclein, recapitulate certain hallmarks of abnormally processed protein biology (e.g., alpha synuclein biology). This discovery permits the carrying out of screens in yeast to identify compounds that modulate abnormal biological processes that occur in the yeast as a result of the expression of the abnormally processed protein (e.g., alpha synuclein). Compounds identified by such screens can be used as candidate drugs for the treatment of prevention of protein misfolding diseases such as Parkinson's Disease.

The invention is also based, at least in part, on the discovery that yeast cells having a certain dosage of a nucleic acid encoding alpha synuclein exhibit growth defects proportional to the amount of alpha synuclein present in the yeast. Moderate growth defects were observed in yeast having one integrated copy of an alpha synuclein-encoding nucleic acid, whereas extreme growth defects were observed in yeast having two integrated copies of such a nucleic acid. Such yeast provide excellent systems for identifying compounds that suppress or inhibit alpha synuclein-induced toxicity. Compounds identified by such screens can also be used as candidate drugs for the treatment of prevention of protein misfolding diseases such as Parkinson's Disease.

Described herein are yeast cells that ectopically express a protein (e.g., alpha-synuclein) associated with a protein misfolding disease or condition, such as a neurologic disorder or neurodegenerative condition in humans. Such a protein undergoes abnormal processing in human cells (e.g., neuronal cells), resulting in abnormal distribution within the cells (e.g., intracellular, such as intraneuronal inclusions or membrane localization), aggregate formation in the cells, and/or toxicity to cells. Such proteins are referred to herein as “abnormally processed proteins” and the associated diseases, conditions, or disorders as “protein misfolding diseases.” Yeast cells expressing such a protein, which can be a wildtype or a mutant protein or a functional variant thereof, are useful for identifying drugs which inhibit misfolding and/or abnormal processing of proteins and, thus, are useful for prevention and/or treatment (including inhibition of progression and reversal) of protein misfolding diseases. Such diseases include, for example, Parkinson's Disease, Parkinson's Disease with accompanying dementia, dementia with Lewy bodies, Alzheimer's Disease, Alzheimer's Disease with Parkinsonism, multiple system atrophy, Huntington's Disease, and spinocerebellar ataxia.

In some embodiments, a protein (e.g., alpha-synuclein) which is characteristic of a protein misfolding disease (e.g., Parkinson's Disease, dementia with Lewy bodies, or multiple system atrophy) is ectopically expressed in yeast cells. The resulting yeast cells are useful both for identifying drugs (compounds or molecules) that inhibit (partially or totally) abnormal processing of the protein and for identifying genetic targets which can be modulated to inhibit (partially or totally) abnormal processing of the protein. In further embodiments, for example, Tau protein, which forms neurofibrillary tangles, Huntingtin (Htt) protein with expanded Q repeats, or ataxin with expanded Q repeats is ectopically expressed in yeast. The resulting yeast cells are useful, respectively, for identifying drugs useful in therapy of Alzheimer's Disease, Huntington's Disease and spinocerebellar ataxias.

Yeast can be used as a model for studying neurologic disorders in whose etiology alpha-synuclein plays a role, as well as for identifying drugs (compounds or molecules) that interfere with localization of alpha-synuclein to cell membranes and/or formation of alpha-synuclein cytoplasmic inclusions and, thus, for identifying drugs for use in inhibiting the adverse effects of aS. Specific embodiments relate to a yeast model useful for identifying drugs that inhibit alpha-synuclein and are useful for treating conditions in whose etiology alpha-synuclein plays a role (preventing or delaying the onset, reducing the extent to which a condition occurs and/or reversing the condition once it has occurred). For example, yeast systems described herein can be used to identify drugs for use in therapy of neurodegenerative disorders, such as Parkinson's Disease, Parkinson's Disease with accompanying dementia, Lewy body dementia, and Alzheimer's disease with Parkinsonism Lewy. Such drugs can be used to prevent or delay the onset, reduce the extent of occurrence or reverse conditions characterized by inclusions mainly reactive for alpha-synuclein.

The yeast systems described herein comprise yeast cells, such as Saccharomyces (e.g., S. cerevisiae) cells which ectopically express an abnormally processed protein. The ectopically-expressed protein can be a mammalian protein (e.g., human or mouse), and can be wildtype or mutant or a functional variant thereof. In particular embodiments, a protein described herein is expressed ectopically in yeast. Yeast cells described herein which ectopically express wildtype or mutant (or a functional variant thereof) abnormally processed protein have incorporated therein nucleic acids (DNA or RNA) encoding the abnormally processed protein.

In one aspect, yeast cells, such as S. cerevisiae, comprise (have incorporated therein) a plasmid or plasmids that comprise DNA or RNA encoding the abnormally processed protein (e.g., alpha-synuclein) that is expressed. In one embodiment, DNA encoding the abnormally processed protein is operably linked to a promoter that is functional in yeast, such as a promoter functional in S. cerevisiae (e.g., Gal 1-10 promoter). A wide variety of plasmids can be used. In one embodiment, the plasmid is an integrative plasmid (e.g., pRS303, pRS304, pRS305, pRS306, or any other integrative plasmids). In further embodiments, the plasmid is an episomal plasmid (e.g., p426GPD, p416GPD, p426TEF, p423GPD, p425GPD, p424GPD or p426GAL).

Yeast cells described herein can express a fusion protein, referred to as an abnormally processed protein (e.g., alpha-synuclein)-detectable protein fusion protein. In one embodiment, DNA encoding an abnormally processed protein (wildtype or mutant or a functional variant thereof) and DNA encoding the detectable protein are in frame in a plasmid capable of expression (which is expressed) in yeast, with the result that the expressed product is a fusion protein. The two components can be operably linked in the plasmid to one promoter, which drives their expression, or each may be operably linked to a different (separate) promoter, provided that the desired fusion protein is produced. Optionally, the two components may be separated by intervening residues, for example, a linker polypeptide that may enable the fusion protein to attain slightly different properties. The detectable protein can be, for example, a fluorescent protein, an enzyme or an epitope. The fluorescent protein can be, for example, a red fluorescent protein, a green fluorescent protein, a blue fluorescent protein, a yellow fluorescent protein, a cyan fluorescent protein, or other variants of the fluorescent proteins. Enzymes components of the fusion proteins can be, for example, beta-galactosidase, luciferase, Ura3p or other auxotrophic marker proteins. Epitope components can be, for example, FLAG, HA, His6, AU1, Tap, Protein A, or other epitope. In specific embodiments, yeast cells that ectopically express an abnormally processed protein, alone or as a component of a fusion protein, further comprise at least one gene that plays an important role in drug efflux and/or cell permeability that has been disrupted (rendered nonfunctional). Such disrupted genes include, but are not limited to, the PDR1 gene, the PDR3 gene, the PDR5 gene, and the ERG6 gene.

One embodiment features a yeast cell comprising an expression construct (e.g., a plasmid described herein) comprising a nucleic acid encoding a protein comprising an alpha synuclein, wherein the expression construct is integrated in the genome of the yeast cell, and wherein expression of the nucleic acid is regulated by an inducible promoter (e.g., an inducible promoter described herein), such that induction of production of the protein is toxic to the yeast cell. The yeast cell can optionally comprise two or more integrated copies of the expression construct.

Another embodiment features a yeast cell expressing a toxicity-inducing amount of a protein comprising an alpha synuclein. Such a yeast cell can comprise at least one integrated expression construct (and optionally two or more copies) comprising a nucleic acid encoding the protein.

Alpha synuclein expressed in the yeast described herein can be, for example, human alpha synuclein (e.g., wild type human alpha synuclein or a mutant human alpha-synuclein described herein).

In some embodiments, the yeast cell expressing an alpha synuclein-containing protein is Saccharomyces cerevisiae, Saccharomyces uvae, Saccharomyces kluyveri, Schizosaccharomyces pombe, Kluyveromyces lactis, Hansenula polymorpha, Pichia pastoris, Pichia methanolica, Pichia kluyveri, Yarrowia lipolytica, Candida sp., Candida utilis, Candida cacaoi, Geotrichum sp., or Geotrichum fermentans.

The inducible promoter used in the yeast described herein can be, for example, GAL1-10, GAL1, GALL, GALS, GPD, ADH, TEF, CYC1, MRP7, MET25, TET, VP16, or VP16-ER.

The expression construct used in the yeast described herein can be an integrative plasmid such as pRS303, pRS304, pRS305, or pRS306.

In some embodiments, induction of expression of the nucleic acid renders the yeast cell non-viable and/or arrests growth of the cell.

The alpha synuclein-containing protein can be a fusion protein comprising a detectable protein (e.g., a fluorescent protein, an enzyme, or an epitope). Exemplary fluorescent proteins include a red fluorescent protein, green fluorescent protein, blue fluorescent protein, yellow fluorescent protein, and cyan fluorescent protein.

In some embodiments, at least one gene (e.g., PDR1, PDR3, or ERG6 or PDR5) that encodes a polypeptide involved in drug efflux or cell permeability is disrupted.

Also disclosed is a method of identifying a drug that prevents or suppresses toxicity of alpha-synuclein in cells. The method comprises: (a) contacting a yeast cell (e.g., S. cerevisiae) which ectopically expresses alpha-synuclein with a candidate drug; (b) culturing the yeast cell under conditions that allow for expression of alpha-synuclein at a level sufficient to cause toxicity to the yeast cell; and (c) determining whether the toxicity of alpha-synuclein is less in the presence of the candidate drug than in the absence of the candidate drug, wherein if the toxicity of alpha-synuclein is less in the presence of the candidate drug, a drug that prevents or suppresses the toxicity of alpha-synuclein has been identified. Optionally, mutant alpha-synuclein proteins (e.g., A53T or A30P) can be used in this method.

Another embodiment features a method of identifying a compound that prevents or suppresses alpha-synuclein-induced toxicity, the method comprising: (a) culturing a yeast cell described herein (e.g., a yeast cell comprising two integrated copies of a nucleic acid encoding a protein comprising alpha synuclein) in the presence of a candidate agent and under conditions that allow for expression of the protein at a level that, in the absence of the candidate agent, is sufficient to induce toxicity in the yeast cell; and (b) determining whether toxicity in the yeast cell is less in the presence of the candidate agent as compared to in the absence of the candidate agent, wherein if the toxicity is less in the presence of the candidate agent, then the candidate agent is identified as a compound that prevents or suppresses alpha-synuclein-induced toxicity.

Another aspect is a method of identifying an extragenic suppressor of alpha-synuclein-induced toxicity, the method comprising: (a) culturing a yeast cell described herein (e.g., a yeast cell comprising two integrated copies of a nucleic acid encoding a protein comprising alpha synuclein), wherein an endogenous gene of the yeast cell has been disrupted, under conditions that allow for expression of the alpha synuclein-containing protein at a level that, in the absence of the disruption of the endogenous gene, is sufficient to induce toxicity in the yeast cell; and (b) determining whether toxicity in the yeast cell is less in the presence of the disruption of the endogenous gene as compared to in the absence of the disruption of the endogenous gene, wherein if the toxicity is less in the presence of the disruption of the endogenous gene, then the disrupted endogenous gene is identified as an extragenic suppressor of alpha-synuclein-induced toxicity.

A further embodiment relates to methods in which yeast ectopically expressing an abnormally processed protein such as alpha synuclein (alone or as a component of a fusion protein, as described herein) are used to identify or produce drugs (compounds or molecules) that inhibit abnormal processing of the protein (e.g., alpha-synuclein), such as drugs that inhibit (partially or completely) localization to a cell membrane and/or inhibit (partially or completely) formation of cytoplasmic inclusions/aggregation of the abnormally processed protein and, thus, can be used in treating a neurological or neurodegenerative disorder in which the abnormally processed protein (e.g., alpha-synuclein) contributes to the etiology.

One embodiment features a method of identifying a drug that inhibits localization of an abnormally processed protein (e.g., alpha-synuclein) to a cell membrane. The method comprises (a) culturing yeast cells which ectopically express the abnormally processed protein (e.g., alpha-synuclein) at a low level in the presence of a candidate drug; (b) determining the extent to which the abnormally processed protein associates with yeast plasma membrane and (c) comparing the extent determined in (b) with the extent to which the abnormally processed protein localizes to yeast plasma membrane in an appropriate control, wherein if the extent determined in (b) is less than the extent to which the abnormally processed protein localizes to yeast plasma membrane in the control, the candidate drug is a drug that inhibits localization of the abnormally processed protein to a cell membrane. A control can be, for example, yeast cells that are the same as the test cells and are treated the same as test cells except that they are cultured in the absence of the candidate drug. The control can be carried out at the same time as the test cells or can be a pre-established control, such as a standard curve.

Also disclosed is a method of identifying a compound that modulates alpha-synuclein localization to a plasma membrane, the method comprising: (a) culturing, in the presence of a candidate agent, a yeast cell ectopically expressing a protein comprising an alpha-synuclein; and (b) determining whether localization of the protein to the plasma membrane in the yeast cell is altered in the presence of the candidate agent as compared to in the absence of the candidate agent, wherein if localization of the protein to the plasma membrane is altered in the presence of the candidate agent, then the candidate agent is identified as a compound that modulates alpha-synuclein localization to the plasma membrane.

One embodiment features a method of identifying a drug that inhibits (partially or completely) formation of inclusions/aggregation of an abnormally processed protein (e.g., alpha-synuclein) in cells. The method comprises (a) culturing yeast cells, which ectopically expresses the abnormally processed protein (e.g., alpha-synuclein) at a level sufficient to form inclusions/result in aggregation of the abnormally processed protein in yeast cells, in the presence of a candidate drug; (b) determining the extent to which the abnormally processed protein forms inclusion/aggregates in the yeast cytoplasm and (c) comparing the extent determined in (b) with the extent to which the abnormally processed protein forms inclusions/aggregates in yeast cytoplasm in an appropriate control, wherein if the extent determined in (b) is less than the extent to which the abnormally processed protein forms inclusions/aggregates in yeast cytoplasm in the control, the candidate drug is a drug that inhibits formation of inclusions/aggregation of the abnormally processed protein in cells. A control can be, for example, yeast cells that are the same as the test cells e.g., yeast cells expressing wildtype or mutant alpha-synuclein) and are treated the same as test cells except that they are cultured in the absence of the candidate drug. The control can be carried out at the same time as the test cells or can be a pre-established control, such as a standard curve.

Also disclosed is a method of identifying a compound that inhibits the aggregation or formation of inclusions of alpha-synuclein, the method comprising: (a) culturing, in the presence of a candidate agent, a yeast cell ectopically expressing a protein comprising an alpha-synuclein; and (b) determining whether cytoplasmic aggregation or inclusion formation of the protein is less in the presence of the candidate agent as compared to in the absence of the candidate agent, wherein if aggregation or formation of inclusions of the protein is less in the presence of the candidate agent, then the candidate agent is identified as a compound that inhibits the aggregation or formation of inclusions of alpha-synuclein.

One embodiment features a method of identifying a drug that promotes disaggregation of alpha-synuclein (e.g., promotes reversal of aggregation that has already occurred, such as by causing degradation or disintegration of inclusions or aggregates). The method comprises (a) contacting a yeast cell which ectopically expresses alpha-synuclein, wherein alpha-synuclein has formed inclusions or aggregated in the yeast cytoplasm, with a candidate drug, under conditions suitable for or which result in entry of the candidate drug into the yeast; and (b) determining whether the inclusions or aggregation is less in the presence of the candidate drug than in the absence of the candidate drug, wherein if the inclusions or aggregation occurs is less in the presence of the candidate drug, a drug that promotes disaggregation of alpha-synuclein has been identified. The alpha-synuclein can be, for example, wild type or mutant (e.g., A53T) and a wide variety of yeast cells, such as those described herein, can be used. The wildtype or mutant alpha-synuclein can be expressed alone or as a component of a fusion protein, as also described herein.

A further embodiment features a method of assessing the ability of a compound to promote disaggregation of alpha-synuclein in cells. The method comprises (a) contacting a yeast cell which ectopically expresses alpha-synuclein and comprises alpha-synuclein aggregates in the cytoplasm with a compound to be assessed, under conditions suitable for or which result in entry of the compound into yeast; (b) maintaining the product of (a) for sufficient time for the compound to interact with alpha-synuclein aggregates in the cytoplasm; and (c) determining whether the occurrence of alpha-synuclein aggregates in the cytoplasm of the yeast cell is less in the presence of the compound than in its absence, wherein if the occurrence is less in the presence of the compound than in its absence, the compound promotes disaggregation of alpha-synuclein in cells. The alpha-synuclein can be wild type or mutant (e.g., A53T) and a wide variety of yeast cells, such as those described herein, can be used. The wildtype or mutant alpha-synuclein can be expressed alone or as a component of a fusion protein, as also described herein.

Also disclosed is a method of identifying a compound that promotes disaggregation of alpha-synuclein, the method comprising: (a) providing a yeast cell ectopically expressing a protein comprising an alpha-synuclein, wherein the cell comprises cytoplasmic aggregates or inclusions of the protein; (b) contacting the yeast cell with a candidate agent; and (c) determining whether cytoplasmic aggregation or inclusion formation of the protein is reduced in the presence of the candidate agent as compared to in the absence of the candidate agent, wherein if aggregation or formation of inclusions of the protein is reduced in the presence of the candidate agent, then the candidate agent is identified as a compound that promotes disaggregation of alpha-synuclein.

One embodiment features a method of identifying a drug that prevents or suppresses proteasomal impairment caused by alpha-synuclein in cells. The method comprises: (a) contacting a yeast cell which ectopically expresses alpha-synuclein with a candidate drug; (b) culturing the yeast cell (e.g., S. cerevisiae) under conditions that allow for expression of alpha-synuclein at a level sufficient to cause proteasomal impairment; and (c) determining whether the proteasomal impairment by alpha-synuclein is less in the presence of the candidate drug than in the absence of the candidate drug, wherein if the proteasomal impairment by alpha-synuclein is less in the presence of the candidate drug, a drug that prevents or suppresses proteasomal impairment caused by alpha-synuclein has been identified. Optionally, mutant alpha-synuclein proteins (e.g., A53T, A30P) can be used in this method.

Also disclosed is a method of identifying a compound that prevents or suppresses proteasomal impairment caused by alpha-synuclein, the method comprising: (a) culturing, in the presence of a candidate agent, a yeast cell ectopically expressing a protein comprising an alpha-synuclein; and (b) determining whether proteasomal impairment in the cell is less in the presence of the candidate agent as compared to in the absence of the candidate agent, wherein if proteasomal impairment in the cell is less in the presence of the candidate agent, then the candidate agent is identified as a compound that prevents or suppresses proteasomal impairment caused by alpha-synuclein.

One embodiment features a method of identifying a drug that prevents or suppresses phospholipase D (PLD) inhibition caused by alpha-synuclein in cells. The method comprises: (a) contacting a yeast cell (e.g., S. cerevisiae) which ectopically expresses alpha-synuclein with a candidate drug; (b) culturing the yeast cell under conditions that allow for expression of alpha-synuclein at a level sufficient to cause PLD inhibition; and (c) determining whether the PLD inhibition by alpha-synuclein is less in the presence of the candidate drug than in the absence of the candidate drug, wherein if the PLD inhibition by alpha-synuclein is less in the presence of the candidate drug, a drug that prevents or suppresses PLD inhibition caused by alpha-synuclein has been identified. Optionally, mutant alpha-synuclein proteins (e.g., A53T, A30P) can be used in this method.

Also disclosed is a method of identifying a compound that prevents or suppresses PLD inhibition caused by alpha-synuclein, the method comprising: (a) culturing, in the presence of a candidate agent, a yeast cell ectopically expressing a protein comprising an alpha-synuclein; and (b) determining whether PLD inhibition in the cell is less in the presence of the candidate agent as compared to in the absence of the candidate agent, wherein if PLD inhibition in the cell is less in the presence of the candidate agent, then the candidate agent is identified as a compound that prevents or suppresses PLD inhibition caused by alpha-synuclein.

One embodiment features a method of identifying a drug that prevents or suppresses oxidative stress caused by alpha-synuclein in cells. The method comprises: (a) contacting a yeast cell (e.g., S. cerevisiae) which ectopically expresses alpha-synuclein with a candidate drug; (b) culturing the yeast cell under conditions that allow for expression of alpha-synuclein at a level sufficient to cause oxidative stress; and (c) determining whether the oxidative stress by alpha-synuclein is less in the presence of the candidate drug than in the absence of the candidate drug, wherein if the oxidative stress by alpha-synuclein is less in the presence of the candidate drug, a drug that prevents or suppresses oxidative stress caused by alpha-synuclein has been identified. Optionally, mutant alpha-synuclein proteins (e.g., A53T, A30P) can be used in this method.

Also disclosed is a method of identifying a compound that prevents or suppresses oxidative stress caused by alpha-synuclein, the method comprising: (a) culturing, in the presence of a candidate agent, a yeast cell ectopically expressing a protein comprising an alpha-synuclein; and (b) determining whether oxidative stress in the cell is less in the presence of the candidate agent as compared to in the absence of the candidate agent, wherein if oxidative stress in the cell is less in the presence of the candidate agent, then the candidate agent is identified as a compound that prevents or suppresses oxidative stress caused by alpha-synuclein.

One embodiment features a method of identifying a drug that modulates interaction of alpha-synuclein with an alpha-synuclein associated protein. The method comprises: (a) contacting a yeast cell (e.g., S. cerevisiae) which ectopically expresses alpha-synuclein and an alpha-synuclein associated protein, with a candidate drug; (b) incubating the yeast cell under conditions that allow for interaction between alpha-synuclein and the alpha-synuclein associated protein; and (c) determining whether the interaction between the two molecules is less in the presence of the candidate drug than in the absence of the candidate drug, wherein if the interaction is less in the presence of the candidate drug, a drug that reduces or inhibits interaction of alpha-synuclein with an alpha-synuclein associated protein has been identified. Optionally, mutant alpha-synuclein proteins (e.g., A53T, A30P) can be used in this method.

Also disclosed is a method of identifying a compound that reduces or inhibits an interaction of alpha-synuclein with an alpha-synuclein associated protein, the method comprising: (a) culturing, in the presence of a candidate agent, a yeast cell ectopically expressing (i) a first protein comprising an alpha-synuclein, and (ii) a second protein comprising an alpha-synuclein associated protein; and (b) determining whether the interaction between the first protein and the second protein in the cell is less in the presence of the candidate agent as compared to in the absence of the candidate agent, wherein if the interaction between the first protein and the second protein in the cell is less in the presence of the candidate agent, then the candidate agent is identified as a compound that reduces or inhibits the interaction of alpha-synuclein with the alpha-synuclein associated protein. Exemplary alpha-synuclein associated proteins include dephospho-BAD, protein kinase C (PKC), mitogen-activated extracellular regulated kinase (ERK), synphilin-1, Huntingtin (Htt), phospholipase D (PLD), parkin, and Tau.

One embodiment features a method of identifying an alpha-synuclein associated protein. The method comprises: (a) transforming a yeast cell (e.g., S. cerevisiae) with plasmids encoding alpha-synuclein and a candidate protein; (b) incubating the yeast cell under conditions that allow for expression of alpha-synuclein and the candidate protein; and (c) determining the interaction between alpha-synuclein and the candidate protein, wherein if the interaction occurs, the candidate protein is an alpha-synuclein associated protein. Optionally, mutant alpha-synuclein proteins (e.g., A53T, A30P) can be used in this method.

Also disclosed is a method of identifying an alpha-synuclein associated protein, the method comprising: (a) transforming a yeast cell with (i) a first expression construct encoding a first protein comprising an alpha-synuclein, and (ii) a second expression construct encoding a second protein comprising a candidate protein; (b) incubating the yeast cell under conditions that allow for expression of the first protein and the second protein; and (c) determining whether the first protein interacts with the second protein, wherein if an interaction occurs, the candidate protein is identified as an alpha-synuclein associated protein.

One embodiment features a method of identifying a gene that is involved in an alpha-synuclein associated disease. The method comprises: (a) isolating RNAs from a yeast cell (e.g., S. cerevisiae) that ectopically expresses alpha-synuclein; (b) isolating RNAs from a control yeast cell; (c) comparing the expression patterns of the RNAs isolated from (a) and (b); and (d) identifying genes that are expressed at higher or lower levels in the yeast cell that ectopically expresses alpha-synuclein relative to the control yeast cell. The method may be carried out by performing differential display (e.g., microarray) or subtractive hybridization. Optionally, mutant alpha-synuclein proteins (e.g., A53T, A30P) can be used in this method. Examples of the alpha-synuclein associated disease include Parkinson's Disease, Parkinson's Disease with accompanying dementia, Lewy body dementia, Alzheimer's disease with Parkinsonism, and multiple system atrophy.

Also disclosed is a method of identifying a gene that is involved in an alpha-synuclein associated disease, the method comprising: (a) isolating RNAs from a first yeast cell that ectopically expresses alpha-synuclein; (b) isolating RNAs from a second yeast cell that does not ectopically express alpha-synuclein; (c) comparing the RNAs isolated from the first yeast cell and the RNAs isolated from the second yeast cell; and (d) identifying an RNA that is present at a higher or lower level in the first cell relative to the second yeast cell, to thereby identify a corresponding gene that is involved in an alpha-synuclein associated disease. In such a method, the RNA can be identified, for example, by performing differential display or subtractive hybridization.

One embodiment features a method of identifying a drug that inhibits cells from developing Lewy pathology, comprising (a) culturing yeast cells, such as S. cerevisiae cells, which ectopically expresses an abnormally processed protein associated with formation of Lewy pathology (test cells), at a level sufficient to result in aggregation of the protein in yeast cytoplasm in the presence of a candidate drug; (b) determining the extent to which the protein aggregates in the yeast cell cytoplasm; and (c) comparing the extent determined in (b) (in the test cells) with the extent to which the protein aggregates in yeast cytoplasm in an appropriate control, wherein if the extent determined in (b) is less that the extent to which the abnormally processed protein aggregates in cytoplasm in the control, the candidate drug is a drug that inhibits cells from developing Lewy pathology.

The compounds identified by the yeast screens described herein can be tested for their effectiveness in vivo (e.g., in a mammal such as a mouse or a human). An exemplary in vivo model is a transgenic mouse whose genome comprises a transgene that encodes the abnormally processed protein (e.g., alpha synuclein) and is expressed in the mouse (e.g., in neuronal cells) in such a manner that it results in cell toxicity, membrane association of the protein, formation of inclusions, and/or inhibition of PLD.

Also disclosed are assay kits comprising yeast cells described herein.

A further embodiment relates to a method of treating an individual or subject at risk for developing or suffering from a protein misfolding disease (e.g., a neurodegenerative disorder). The method comprises administering a therapeutically effective amount of a compound (e.g., drug) to the subject, wherein the compound (e.g., drug) was identified by any method as described herein. The identified compound can be a small molecule compound, a peptidomimetic, a nucleic acid, or a polypeptide. The identified compound can be a natural product, synthetic compound, or semi-synthetic compound. Optionally, the compound for treatment is formulated with a pharmaceutically acceptable carrier. The compound can be administered alone or in combination with another method or methods of treating such an individual (e.g., in combination with another drug, surgery or stem cell or neuronal cell implantation). Examples of the protein misfolding disease include Parkinson's disease, Parkinson's Disease with accompanying dementia, dementia with Lewy bodies, Alzheimer's Disease, Alzheimer's Disease with Parkinsonism, multiple system atrophy (MSA), Huntington's Disease, spinocerebellar ataxia (SCA), prion diseases, and type 2 diabetes.

One example features a method of treating an individual suffering from a protein misfolding disease (e.g., Parkinson's Disease), the method comprising administering to the individual a pharmaceutical composition comprising a therapeutically effective amount of a compound identified by a method described herein.

As is clear from work described herein, yeast that ectopically express an abnormally processed protein, such as alpha synuclein, recapitulate hallmarks of abnormally processed protein biology (e.g., alpha-synuclein biology), such as: 1) membrane association; 2) formation of inclusions; 3) differences in the behavior of wildtype and A53T versus A30P; 4) ubiquitination; 5) toxicity; 6) interactions with mutant huntingtin (htt); 7) oxidative stress; and 8) inhibition of PLD.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, the preferred methods and materials are described below. All publications, patent applications, patents, and other references mentioned herein are incorporated by reference in their entirety. In case of conflict, the present application, including definitions, will control. In addition, the materials, methods, and examples are illustrative only and not intended to be limiting.

Other features and advantages of the invention will be apparent from the following detailed description, and from the claims.

### BRIEF DESCRIPTION OF THE DRAWINGS

FIGS. 1A-1B depict aS localization to the plasma membrane of yeast cells. YFP control (1A) or wild type aS fused to YFP (1B) were expressed in the yeast cytoplasm.

FIGS. 2A-2D show that aggregation of aS occurs with the WT (2B) and A53T (2C) mutant but not with the A30P (2D) or GFP control (2A). Alpha synuclein was fused to GFP and expressed in the yeast cytoplasm.

FIG. 3 shows the toxicity phenotype observed upon over-expression of alpha-synuclein-GFP fusions in the yeast cytoplasm. Cells were serially diluted (5-fold at each step) and spotted onto glucose or galactose medium.

FIGS. 4A-4D show co-aggregation of alpha-synuclein and PQ103. In cells co-expressing aS and Q25, aS shows its normal distribution where some cells show inclusions and others show membrane association. In these cells, Q25 is soluble. Cells co-expressing aS and Q103 show tight co-localization of both proteins in cytoplasmic inclusions.

### DETAILED DESCRIPTION OF THE INVENTION

Yeast ectopically expressing an abnormally processed protein (e.g., alpha-synuclein) can be used to study diseases associated with abnormally processed proteins. As used herein, the term “abnormally processed protein” refers to a protein that undergoes abnormal processing in human cells (e.g., neuronal cells), resulting in abnormal distribution (e.g., intracellular, such as intraneuronal, inclusions or membrane localization), aggregate formation and/or toxicity to the cells. Any disease associated with an abnormally processed protein is referred to herein as a “protein misfolding disease.”

Protein misfolding, protein fibril formation, and/or protein aggregation may contribute to numerous neurodegenerative diseases (e.g., Parkinson's disease, Parkinson's Disease with accompanying dementia, dementia with Lewy bodies, Alzheimer's Disease, Alzheimer's Disease with Parkinsonism, multiple system atrophy (MSA), Huntington's Disease, spinocerebellar ataxia (SCA), and prion diseases) as well as non-neuronal diseases (e.g., type 2 diabetes). Yeast cells that ectopically expressing such a protein, which can be a wildtype or a mutant protein or a functional variant thereof, are useful for identifying candidate drugs which inhibit misfolding and/or abnormal processing of proteins and, thus, are useful for prevention and/or treatment (including inhibition of progression and reversal) of protein misfolding diseases.

Parkinson's disease (PD) is one example of protein misfolding diseases. Studies of the genetic basis of PD identified two missense mutations in the alpha-synuclein gene (Kruger R, et al., 1998. Nat. Genet. 18, 106-108; Polymeropoulos M H, et al., 1997. Science 276, 2045-2047). One of these mutations is a substitution of an alanine for a threonine at position 53 (A53T), the other is an alanine for a proline at position 30 (A30P). Alpha synuclein was the first “PD gene” to be discovered, and it may also be involved in the pathogenesis of other neurodegenerative diseases, such as Alzheimer's disease and multiple system atrophy. Although the normal cellular role of alpha synuclein is still unidentified, important progress has recently been made both in identifying interacting proteins (alpha synuclein associated proteins) and uncovering the patterns of toxicity. Alpha synuclein associated proteins may include, but are not limited to, dephospho-BAD (a Bcl-2 homologue), protein kinase C (PKC), the mitogen-activated extracellular regulated kinase (ERK), synphilin-1, huntingtin (htt), phospholipase D (PLD), parkin, and Tau.

In certain aspects, the present disclosure relates to compositions and methods for treating protein misfolding diseases. Such diseases include, but are not limited to, Parkinson's Disease, Parkinson's Disease with accompanying dementia, Lewy body dementia, Alzheimer's disease with Parkinsonism, and multiple system atrophy. The present inventors have developed a yeast-based system for identification of candidate therapeutic agents for these diseases, which interfere with the activity or function of an abnormally processed protein such as alpha-synuclein. As described above, alpha-synuclein can associate with many other proteins, such as dephospho-BAD, protein kinase C (PKC), mitogen-activated extracellular regulated kinase (ERK), synphilin-1, huntingtin (htt), phospholipase D (PLD), parkin, and Tau. Accordingly, one aspect of the disclosure contemplates expression of these alpha synuclein associated proteins in the yeast based system.

Although the present disclosure is exemplified with reference to yeast cells which ectopically express alpha-synuclein, methods and compositions described herein may be also used to identifying drugs useful for therapy of protein misfolding diseases in which the abnormally processed protein is other than alpha-synuclein, such as amyloid-β (Alzheimer's disease), Tau (Alzheimer's disease), huntingtin (Huntington's disease), PrP (prion diseases), and islet amyloid polypeptide (type 2 diabetes).

A specific embodiment described herein is a yeast model useful for studying neurodegenerative disorders in whose etiology alpha-synuclein (aS) plays a role, as well as for identifying drugs (compounds or molecules) that inhibit, for example, aS-induced toxicity, localization of aS to cell membranes, and/or formation of aS cytoplasmic inclusions and, thus, for identifying drugs for use in inhibiting the adverse effects of aS. In specific embodiments, the present disclosure relates to a yeast model useful for identifying drugs that inhibit aS and are useful for treating conditions in whose etiology aS plays a role (preventing or delaying the onset, reducing the extent to which a condition occurs and/or reversing the condition once it has occurred). For example, the yeast system described herein can be used to identify drugs for use in therapy of neurodegenerative disorders, such as Parkinson's Disease, Parkinson's Disease with accompanying dementia, Lewy body dementia and Alzheimer's disease with Parkinsonism Lewy. Such drugs can be used to prevent or delay the onset, reduce the extent of occurrence or reverse conditions in which alpha-synuclein (wildtype or mutant) is abnormally processed or misfolded.

Disclosed herein are yeast cells (e.g., S. cerevisiae) which ectopically express an abnormally processed protein (e.g., alpha-synuclein). The ectopically-expressed abnormally processed protein can be wildtype or mutant or a functional variant thereof. In certain embodiments, yeast cells express an aS protein in one of the two forms: (1) aS (or a fragment or variant of aS) which is not a component of a fusion protein (e.g., not a component of a chimeric product that includes non-aS amino acid sequences); or (2) aS (or a fragment or variant of aS) as a component of a fusion protein which includes aS and non-aS amino acid sequences such as amino acids sequences encoding a detectable protein (peptide or polypeptide). For example, the detectable protein (peptide or polypeptide) can be a fluorescent protein, an epitope or an enzyme.

### Yeast Cells

After several decades of intense study, yeast (e.g., Saccharomyces cerevisiae) has become an extraordinarily powerful system for studying complex biological problems. There are numerous advantaged to using yeast as a model system. These include: 1) switching readily between haploid and diploid genetics; 2) the ease of site directed mutagenesis; 3) the availability of many expression vectors; 4) methods for genetic and chemical screens that can be performed at a fraction of the price in time and materials required in other systems; 5) a chaperone machinery, particularly relevant for problems involving protein folding, that is extensively characterized; and 6) special strains with greatly enhanced drug sensitivities. Finally, because the yeast genome was the first eukaryotic genome to be sequenced it is currently the single best-characterized eukaryotic cell.

Described herein are experimental results demonstrating that yeast cells ectopically expressing aS faithfully recapitulate many aspects of aS biology and can therefore be used to investigate Parkinson's Disease. In particular, yeast have been shown to be a useful model system or living test tubes for studying protein misfolding.

Yeast strains that can be used in the compositions and methods described herein include, but are not limited to, Saccharomyces cerevisiae, Saccharomyces uvae, Saccharomyces kluyveri, Schizosaccharomyces pombe, Kluyveromyces lactis, Hansenula polymorpha, Pichia pastoris, Pichia methanolica, Pichia kluyveri, Yarrowia lipolytica, Candida sp., Candida utilis, Candida cacaoi, Geotrichum sp., and Geotrichum fermentans. Although much of the discussion herein relates to Saccharomyces cerevisiae which ectopically expresses an abnormally processed protein, this is merely for illustrative purposes. Other yeast strains can be substituted for S. cerevisiae.

Certain aspects of the disclosure relate to screening methods for identifying candidate therapeutic agents (e.g., pharmaceutical, chemical, or genetic agents). The methods described herein can be carried out in yeast strains bearing mutations in the ERG6 gene, the PDR1 gene, the PDR3 gene, the PDR5 gene, and/or any other gene which affects membrane efflux pumps and/or increases permeability for drugs.

### Alpha Synuclein Proteins

In certain aspects, compositions and methods disclosed herein use a protein comprising an alpha synuclein polypeptide. Optionally, the compositions and methods contemplate the use of other proteins involved in alpha synuclein associated diseases and/or protein misfolding diseases, for example, an alpha synuclein-associated protein.

The term “alpha synuclein” encompasses naturally occurring alpha synuclein sequences (e.g., naturally occurring wild type and mutant alpha synucleins) as well as functional variants thereof.

Human alpha synuclein is encoded by the following nucleotide sequence:

(SEQ ID NO: 1)
atggatgtattcatgaaaggactttcaaaggccaaggagggagttgtgg
ctgctgctgagaaaaccaaacagggtgtggcagaagcagcaggaaagac
aaaagagggtgttctctatgtaggctccaaaaccaaggagggagtggtg
catggtgtggcaacagtggctgagaagaccaaagagcaagtgacaaatg
ttggaggagcagtggtgacgggtgtgacagcagtagcccagaagacagt
ggagggagcagggagcattgcagcagccactggctttgtcaaaaaggac
cagttgggcaagaatgaagaaggagccccacaggaaggaattctggaag
atatgcctgtggatcctgacaatgaggcttatgaaatgccttctgagga
agggtatcaagactacgaacctgaagcctaa.

Human alpha synuclein has the following amino acid sequence:

(SEQ ID NO: 2)
MDVFMKGLSKAKEGVVAAAEKTKQGVAEAAGKTKEGVLYVGSKTKEGVV
HGVATVAEKTKEQVTNVGGAVVTGVTAVAQKTVEGAGSIAAATGFVKKD
QLGKNEEGAPQEGILEDMPVDPDNEAYEMPSEEGYQDYEPEA.

The term “variants” is used herein to include functional fragments, mutants and derivatives. For example, “variants” may include substitutions of naturally occurring amino acids at specific sites (e.g., conservative amino acid substitutions), including but not limited to, naturally and non-naturally occurring amino acids. In some embodiments, an alpha synuclein protein is at least 80%, 85%, 90%, 95%, or 98% identical to the amino acid sequence of SEQ ID NO:2 and retains alpha-synuclein function.

As used herein, “activity” or “function” of alpha-synuclein includes, but is not limited to, formation of inclusions/aggregation in the cytoplasm, association with cell membrane, interaction with an alpha-synuclein associated protein. In addition, alpha-synuclein can inhibit PLD activity, cause toxicity to cells, and lead to impaired proteasomal activity.

In some embodiments, a full-length alpha synuclein protein may be used. The term “full-length” refers to an alpha synuclein protein that contains at least all the amino acids encoded by the alpha synuclein cDNA. In other embodiments, different lengths of the alpha synuclein protein may be used. For example, only functionally active domains of the protein may be used. Thus, a protein fragment of almost any length may be employed.

In certain embodiments, variants of the aS protein can be used. Such variants may include biologically-active fragments of the aS protein. These include proteins with aS activity that have amino acid substitutions. In certain embodiments, aS mutants are ectopically expressed in yeast include the A53T mutant (containing a substitution of an alanine for a threonine at position 53) and the A30P mutant (containing a substitution of an alanine for a proline at position 30).

In certain embodiments, fusion proteins including at least a portion of the aS protein may be used. For example, a portion of the aS protein may be fused with a second domain. The second domain of the fusion proteins can be selected from the group consisting of an immunoglobulin element, a dimerizing domain, a targeting domain, a stabilizing domain, and a purification domain. Alternatively, a portion of aS protein can be fused with a heterologous molecule such as a detection protein. Exemplary detection proteins include: (1) a fluorescent protein such as green fluorescent protein (GFP), cyan fluorescent protein (CFP) or yellow fluorescent protein (YFP); (2) an enzyme such as β-galactosidase or alkaline phosphatase (AP); and (3) an epitope such as glutathione-S-transferase (GST) or hemagluttin (HA). To illustrate, an alpha synuclein protein can be fused to GFP at the N- or C-terminus or other parts of the aS protein. These fusion proteins provide methods for rapid and easy detection and identification of the aS protein in the recombinant host cell, exemplified herein by the yeast cell.

In a particular embodiment, the present disclosure contemplates the use of β-synuclein or γ-synuclein proteins in the yeast based system. The synuclein family includes at least three known proteins: α-synuclein, β-synuclein, and γ-synuclein. All synucleins have in common a highly conserved alpha-helical lipid-binding motif with similarity to the class-A2 lipid-binding domains of the exchangeable apolipoproteins (see e.g., George J M, 2002, Genome Biol. 3:S3002). In some embodiments, the disclosure refers to β-synuclein nucleic acid sequence and its corresponding β-synuclein protein sequence by Genbank Accession numbers NM_{—}003085 and NP_{—}003076, respectively.

β-synuclein is encoded by the following nucleotide sequence:

(SEQ ID NO: 3)
atggacgtgttcatgaagggcctgtccatggccaaggagggcgttgtgg
cagccgcggagaaaaccaagcagggggtcaccgaggcggcggagaagac
caaggagggcgtcctctacgtcggaagcaagacccgagaaggtgtggta
caaggtgtggcttcagtggctgaaaaaaccaaggaacaggcctcacatc
tgggaggagctgtgttctctggggcagggaacatcgcagcagccacagg
actggtgaagagggaggaattccctactgatctgaagccagaggaagtg
gcccaggaagctgctgaagaaccactgattgagcccctgatggagccag
aaggggagagttatgaggacccaccccaggaggaatatcaggagtatga
gccagaggcgtag.

β-synuclein has the following amino acid sequence:

(SEQ ID NO: 4)
MDVFMKGLSMAKEGVVAAAEKTKQGVTEAAEKTKEGVLYVGSKTREGVV
QGVASVAEKTKEQASHLGGAVFSGAGNIAAATGLVKREEFPTDLKPEEV
AQEAAEEPLIEPLMEPEGESYEDPPQEEYQEYEPEA.

In some embodiments, the disclosure refers to γ-synuclein nucleic acid sequence and its corresponding γ-synuclein protein sequence by Genbank Accession numbers NM_{—}003087 and NP_{—}003078, respectively. γ-synuclein is encoded by the following nucleotide sequence:

(SEQ ID NO: 5)
atggatgtcttcaagaagggcttctccatcgccaaggagggcgtggtgg
gtgcggtggaaaagaccaagcagggggtgacggaagcagctgagaagac
caaggagggggtcatgtatgtgggagccaagaccaaggagaatgttgta
cagagcgtgacctcagtggccgagaagaccaaggagcaggccaacgcgg
tgagcgaggctgtggtgagcagcgtcaacactgtggccaccaagaccgt
ggcatccaaagagaaagaggaagtggcagaggaggcccagagtggggga
gactag.

γ-synuclein has the following amino acid sequence:

(SEQ ID NO: 6)
MDVFKKGFSIAKEGVVGAVEKTKQGVTEAAEKTKEGVMYVGAKTKENVV
QSVTSVAEKTKEQANAVSEAVVSSVNTVATKTVEEAENIAVTSGVVRKE
DLRPSAPQQEGVASKEKEEVAEEAQSGGD.

Alpha synuclein (aS) Nucleic Acids

Described herein are methods of transferring nucleic acids encoding an aS protein into a yeast cell so that the yeast cell expresses the aS protein. The disclosure also contemplates nucleic acids encoding other proteins that are involved in alpha synuclein associated diseases and/or protein misfolding diseases, such as an alpha synuclein associated protein, or β-synuclein protein.

The term “alpha synuclein nucleic acid” encompasses a nucleic acid comprising a sequence as represented in SEQ ID NO: 1 as well as any of the variants of the alpha synuclein nucleic acid as described herein. The term “variants” is used herein to include all fragments, mutants and derivatives. For example, “variants” may include substitutions of naturally occurring nucleotides at specific sites, including but not limited to naturally and non-naturally occurring nucleotides. Exemplary aS variant nucleic acids include those encoding the A53T and A30P mutant proteins.

In one embodiment, the alpha synuclein nucleic acids encode a full-length or a functional equivalent form of such a protein or polypeptide. In additional embodiments, a truncated polypeptide or a polypeptide with internal deletions is provided to a yeast cell. In certain embodiments, the nucleic acid encodes at least one protein (e.g., aS) or a biologically functional equivalent thereof. In other embodiments, the polypeptide is a human alpha synuclein or other mammalian homologues of alpha synuclein.

In yet other aspects, the disclosure contemplates co-transfecting the yeast cell with any protein that may associate with an alpha synuclein protein. For example, an alpha-synuclein associated protein may be dephospho-BAD, protein kinase C (PKC), mitogen-activated extracellular regulated kinase (ERK), synphilin-1, Huntington (htt), phospholipase D (PLD), parkin, or Tau.

Isolation or creation of at least one recombinant construct or at least one recombinant host cell through the application of recombinant nucleic acid technology is well known to those of skill in the art. The recombinant construct or host cell may comprise at least one nucleic acid encoding a protein involved in alpha synuclein associated diseases and/or protein misfolding diseases, and may express at least one such protein or at least one biologically functional equivalent thereof.

In some embodiments, the disclosure refers to DNA sequences identified by Genbank Accession number NM_{—}000345 for alpha-synuclein nucleic acid. The corresponding aS protein is identified by Genbank Accession number NP_{—}000336.

The term “nucleic acid” generally refers to at least one molecule or strand of DNA, RNA or a derivative or mimic thereof, comprising at least one nucleobase, for example, a naturally occurring purine or pyrimidine base found in DNA or RNA. The term “nucleic acid” encompasses the terms “oligonucleotide” and “polynucleotide.” Generally, the term “nucleic acid” refers to at least one single-stranded molecule, but in specific embodiments will also encompass at least one additional strand that is partially, substantially or fully complementary to the at least one single-stranded molecule. Thus, a nucleic acid may encompass at least one double-stranded molecule or at least one triple-stranded molecule that comprises one or more complementary strand(s) or “complement(s)” of a particular sequence comprising a strand of the molecule.

In certain embodiments, the nucleic acid is a nucleic acid fragment of the aS gene. As used herein, the term “gene” refers to a nucleic acid that is transcribed. The term “nucleic acid fragment” refers to smaller fragments of a nucleic acid, such as those that encode only part of an aS polypeptide sequence. In certain aspects, the gene includes regulatory sequences involved in transcription, or message production or composition.

### Nucleic Acid Vectors for Expression in Yeast

A nucleic acid encoding a component of an assay system described herein (e.g., alpha synuclein, an alpha synuclein associated protein, or any other protein involved in fibril formation and/or in protein aggregation) may be transfected into a yeast cell using nucleic acid vectors that include, but are not limited to, plasmids, linear nucleic acid molecules, artificial chromosomes, and episomal vectors.

Three well known systems used for recombinant plasmid expression and replication in yeast cells include integrative plasmids, low-copy-number ARS-CEN plasmids, and high-copy-number 2μ plasmids. See Sikorski, “Extrachromsomoal cloning vectors of Saccharomyces cerevisiae,” in Plasmid, A Practical Approach, Ed. K. G. Hardy, IRL Press, 1993; and Yeast Cloning Vectors and Genes, Current Protocols in Molecular Biology, Section II, Unit 13.4, Eds., Ausubel et al., 1994.

An example of the integrative plasmids is YIp, which is maintained at one copy per haploid genome, and is inherited in Mendelian fashion. Such a plasmid, containing a gene of interest, a bacterial origin of replication and a selectable gene (typically an antibiotic-resistance marker), is produced in bacteria. The purified vector is linearized within the selectable gene and used to transform competent yeast cells.

An example of the low-copy-number ARS-CEN plasmids is YCp, which contains the autonomous replicating sequence (ARS1) and a centromeric sequence (CEN4). These plasmids are usually present at 1-2 copies per cell. Removal of the CEN sequence yields a YRp plasmid, which is typically present in 100-200 copies per cell. However, this plasmid is both mitotically and meiotically unstable.

An example of the high-copy-number 2μ plasmids is YEp, which contains a sequence approximately 1 kb in length (named the 2μ sequence). The 2μ sequence acts as a yeast replicon giving rise to higher plasmid copy number. However, these plasmids are unstable and require selection for maintenance. Copy number is increased by having on the plasmid a selection gene operatively linked to a crippled promoter.

A wide variety of plasmids can be used in the compositions and methods described herein. In one embodiment, the plasmid is an integrative plasmid (e.g., pRS303, pRS304, pRS305 or pRS306 or other integrative plasmids). In further embodiments, the plasmid is an episomal plasmid (e.g., p426GPD, p416GPD, p426TEF, p423GPD, p425GPD, p424GPD or p426GAL).

Regardless of the type of plasmid used, yeast cells are typically transformed by chemical methods (e.g., as described by Rose et al., 1990, Methods in Yeast Genetics, Cold Spring Harbor Laboratory Press, Cold Spring Harbor, N.Y.). The cells are typically treated with lithium acetate to achieve transformation efficiencies of approximately 10⁴ colony-forming units (transformed cells)/μg of DNA. Yeast perform homologous recombination such that the cut, selectable marker recombines with the mutated (usually a point mutation or a small deletion) host gene to restore function. Transformed cells are then isolated on selective media.

The yeast vectors (plasmids) described herein typically comprise a yeast origin of replication, an antibiotic resistance gene, a bacterial origin of replication (for propagation in bacterial cells), multiple cloning sites, and a yeast nutritional gene for maintenance in yeast cells. The nutritional gene (or “auxotrophic marker”) is most often one of the following: 1) TRP1 (Phosphoribosylanthranilate isomerase); 2) URA3 (Orotidine-5′-phosphate decarboxylase); 3) LEU2 (3-Isopropylmalate dehydrogenase); 4) HIS3 (Imidazoleglycerolphosphate dehydratase or IGP dehydratase); or 5) LYS2 (α-aminoadipate-semialdehyde dehydrogenase).

The yeast vectors (plasmids) described herein may also comprise promoter sequences. A “promoter” is a control sequence that is a region of a nucleic acid sequence at which initiation and rate of transcription are controlled. It may contain genetic elements at which regulatory proteins and molecules may bind, such as RNA polymerase and other transcription factors, to initiate the specific transcription a nucleic acid sequence. The phrases “operatively linked” and “operatively positioned” mean that a promoter is in a correct functional location and/or orientation in relation to a nucleic acid sequence to control transcriptional initiation and/or expression of that sequence.

A promoter may be one naturally associated with a nucleic acid sequence, as may be obtained by isolating the 5′ non-coding sequences located upstream of the coding segment and/or exon. Such a promoter can be referred to as “endogenous.” Alternatively, a promoter may be a recombinant or heterologous promoter, which refers to a promoter that is not normally associated with a nucleic acid sequence in its natural environment. Such promoters may include promoters of other genes and promoters not “naturally occurring.” The promoters employed may be either constitutive or inducible.

For example, various yeast-specific promoters (elements) may be employed to regulate the expression of a RNA in yeast cells. Examples of inducible yeast promoters include GAL1-10, GAL1, GALL, GALS, TET, VP16 and VP16-ER. Examples of repressible yeast promoters include Met25. Examples of constitutive yeast promoters include glyceraldehyde 3-phosphate dehydrogenase promoter (GPD), alcohol dehydrogenase promoter (ADH), translation-elongation factor-1-alpha promoter (TEF), cytochrome c-oxidase promoter (CYC1), and MRP7. Autonomously replicating expression vectors of yeast containing promoters inducible by glucocorticoid hormones have also been described (Picard et al., 1990), including the glucorticoid responsive element (GRE). These and other examples are described in Mumber et al., 1995; Ronicke et al., 1997; Gao, 2000, all incorporated herein by reference. Yet other yeast vectors containing constitutive or inducible promoters such as alpha factor, alcohol oxidase, and PGH may be used. For reviews, see Ausubel et al. and Grant et al., 1987.

To express alpha synuclein proteins in yeast cells, a variety of expression constructs that permitted different levels of expression and different patterns of regulation of aS proteins were generated. For example, 2μ vectors are present in high copy and permit high levels of expression, but they have the disadvantage of varying in number from cell to cell and instability. Integrating constructs are extremely stable but produce lower levels of expression. Constitutive promoters allow expression in normal media, but inducible promoters allow to control the levels and timing of expression. Controllable expression is of particular interest when dealing with potentially toxic proteins, to enhance transformation efficiencies and avoid the accumulation of mutations in the genome that alter aS function and toxicity. Western blotting of aS, A53T, and A30P demonstrated similar levels of accumulation.

### Screening Assays

Certain aspects of the present disclosure provide methods (assays) of screening for a candidate drug (agent or compound) and identifying a drug for treating a protein folding disease. A “candidate drug” as used herein, is any substance with a potential to reduce, interfere with or block activities/functions of an abnormally processed protein (e.g., alpha-synuclein). Various types of candidate drugs may be screened by the methods described herein, including nucleic acids, polypeptides, small molecule compounds, and peptidomimetics. In some cases, genetic agents can be screened by contacting the yeast cell with a nucleic acid construct encoding for a gene. For example, one may screen cDNA libraries expressing a variety of genes, to identify therapeutic genes for the diseases described herein. In other examples, one may contact the yeast cell with other proteins or polypeptides which may confer the therapeutic effect.

As used herein, “activity” or “function” of alpha-synuclein includes, but is not limited to, formation of inclusions/aggregation in the cytoplasm, association with cell membrane, interaction with an aS associated protein. In addition, aS can inhibit PLD activity, cause toxicity to cells, and lead to impaired proteasomal activity.

For example, the identified drugs may prevent protein misfolding, inhibit formation of protein inclusions/aggregation, or promote protein disaggregation. Accordingly, irrespective of the exact mechanism of action, drugs identified by the screening methods described herein will provide therapeutic benefit not only to aS associated diseases, but also to diseases involving protein misfolding or aberrant protein deposition (protein misfolding diseases), including neurodegenerative diseases such as Huntington's, Parkinson's, Alzheimer's, prion-diseases, as well as other non-neuronal diseases such as type 2 diabetes.

In certain embodiments, screening methods described herein use yeast cells that are engineered to express a protein (e.g., an aS protein, an aS associated protein or another protein involved in fibril formation and/or in protein aggregation). For chemical screens, suitable mutations of yeast strains designed to affect membrane efflux pumps and increase permeability for drugs can be used. For example, a yeast strain bearing mutations in the ERG6 gene, the PDR1 gene, the PDR3 gene, and/or the PDR5 gene is contemplated of use. For example, a yeast strain bearing mutations in membrane efflux pumps (erg6, pdr1, pdr3, and/or pdr5) has been successfully used in many screens to identify growth regulators (Jensen-Pergakes K L, et al., 1998. Antimicrob Agents Chemother 42:1160-7).

In certain embodiments, candidate drugs can be screened from large libraries of synthetic or natural compounds. One example is an FDA approved library of compounds that can be used by humans. In addition, synthetic compound libraries are commercially available from a number of companies including Maybridge Chemical Co. (Trevillet, Cornwall, UK), Comgenex (Princeton, N.J.), Brandon Associates (Merrimack, N.H.), and Microsource (New Milford, Conn.), and a rare chemical library is available from Aldrich (Milwaukee, Wis.). Combinatorial libraries are available and can be prepared. Alternatively, libraries of natural compounds in the form of bacterial, fungal, plant and animal extracts are also available, for example, Pan Laboratories (Bothell, Wash.) or MycoSearch (NC), or can be readily prepared by methods well known in the art. It is proposed that compounds isolated from natural sources, such as animals, bacteria, fungi, plant sources, including leaves and bark, and marine samples may be assayed as candidates for the presence of potentially useful pharmaceutical agents. It will be understood that the pharmaceutical agents to be screened could also be derived or synthesized from chemical compositions or man-made compounds. Several commercial libraries can immediately be used in the screens.

Another embodiment relates to a strategy involving “selection” rather than “screening,” and the use of conformationally constrained peptide libraries. For example, Tom Muir of Rockefeller University has developed a system for generating a library of peptides of a defined length based on an intein-catalyzed reaction. Cyclic peptides have many advantages. Like cyclic antibiotics, they have high stability in living cells. In addition, the constrained conformation eliminates the entropic cost of peptide binding, thereby greatly increasing affinities. Work in the Muir lab indicates that this method can be employed in bacterial and mammalian cells; yeast cells also should provide the necessary environment for cyclization. A library with random peptides with a size of seven or nine amino acid residues, in which the first and last are fixed for practical reasons, should generate a very high number of peptides (20⁵=3.2×10⁶ or 20⁷=1.28×10⁹). The library will be created in a yeast expression vector and transformed into yeast cells using control constructs previously established in mammalian and bacterial cells. Because methods disclosed herein select for the restoration of growth in otherwise dying cells, millions of transformants can be analyzed on relatively few plates. Using the double integration stain, the incidence of spontaneous suppressors (false positives) has been found to be negligible.

Another embodiment relates to genetic screens. For example, genomic libraries and disruption libraries can be screened to find extragenic suppressors of aS associated toxicity. Because the yeast genome is small, 10,000 transformants of each type should be sufficient for good coverage. Alternatively, mammalian libraries can be screened.

Potential drugs may include a small molecule. Examples of small molecules include, but are not limited to, small peptides or peptide-like molecules (e.g., a peptidomimetic). As used herein, the term “peptidomimetic” includes chemically modified peptides and peptide-like molecules that contain non-naturally occurring amino acids, peptoids, and the like. Peptidomimetics provide various advantages over a peptide, including enhanced stability when administered to a subject. Methods for identifying a peptidomimetic are well known in the art and include the screening of databases that contain libraries of potential peptidomimetics.

In certain embodiments, such candidate drugs also encompass numerous chemical classes, though typically they are organic molecules, preferably small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl, sulphydryl or carboxyl group.

Other suitable candidate drugs may include antisense molecules, ribozymes, and antibodies (including single chain antibodies), each of which would be specific for the target molecule. For example, an antisense molecule that binds to a translational or transcriptional start site, or splice junctions, would be ideal candidate inhibitors.

One embodiment contemplates screening assays using fluorescent resonance energy transfer (FRET). FRET occurs when a donor fluorophore is in close proximity (10-60 A) to an acceptor fluorophore, and when the emission wavelength of the first overlaps the excitation wavelength of the second (Kenworthy A K et al., 2001. Methods. 24:289-96). FRET should occur when cyan fluorescent protein (CFP) and yellow fluorescent protein (YFP) fusion proteins are actually part of the same complex.

For example, an alpha-synuclein protein is fused to CFP and to YFP respectively, and is integrated in the yeast genome under the regulation of a GAL1-10 promoter. Cells are grown in galactose to induce expression. Upon induction, cells produce the fusion proteins, which aggregate and bring the CFP and YFP close together. Because proteins in the aggregates are tightly packed, the distance between the CFP and YFP is less than the critical value of 100 A that is necessary for FRET to occur. In this case, the energy released by the emission of CFP will excite the YFP, which in turn will emit at its characteristic wavelength. The present inventors contemplate utilizing FRET based screening to identify candidate compounds including, drugs, genes or other factors that can disrupt the interaction of CFP and YFP by maintaining the proteins in a state that does not allow aggregation to occur.

Optionally, interaction of aS with an aS associated protein can be assayed by FRET microscopy, e.g., by fusing alpha-synuclein to CFP and fusing an aS associated protein to YFP. Accordingly, candidate drugs that can modulate the interaction of CFP and YFP can be identified.

One embodiment contemplates screening assays using fluorescence activated cell sorting (FACS) analysis. FACS is a technique well known in the art, and provides the means of scanning individual cells for the presence of fluorescently labeled/tagged moiety. The method is unique in its ability to provide a rapid, reliable, quantitative, and multiparameter analysis on either living or fixed cells. For example, the misfolded aS protein can be suitably labeled, and provide a useful tool for the analysis and quantitation of protein aggregation and fibril and/or aggregate formation as a result of other genetic or growth conditions of individual yeast cells as described above.

In particular embodiments, methods of the present disclosure relate to detecting localization/distribution of aS. An example assay can be carried out in yeast strains in which various cellular compartments have been labeled by YFP fusions to other normal cellular proteins. Co-localization are performed with aS proteins fused to CFP. One skilled in the art would appreciate that results of this evaluation guide the design of biochemical experiments by employing cellular fractionation and co-purification. In addition, results of such assays can determine whether expression of aS alters the normal structure and dynamics of cellular organelles (e.g., Golgi and vesicle trafficking), and provide insight as to normal aS function and putative toxicity mechanisms.

In particular embodiments, methods of the present disclosure relate to determining aS associated toxicity. One of the strongest aspects of yeast is the possibility of performing high throughput screens that may identify genes, peptides and other compounds with the potential to ameliorate toxicity. Yeast systems described herein have the advantage of allowing different types of screens, focusing on different aspects of the biology of aS as readouts. In particular, they will allow for examining changes in cell toxicity, protein aggregation and localization, genetic interactions and proteasome impairment. A large number of compounds can be screened under a variety of growth conditions and in a variety of genetic backgrounds. The toxicity screen has the advantage of not only selecting for compounds that interact with aS, but also upstream or downstream targets that are not themselves cytotoxic and that are not yet identified.

For example, the Bioscreen-C system (Labsystem) permits the growth of up to 200 cell cultures at the same time, under different conditions. Growth rates are monitored optically, recorded automatically, and stored as digital files for further manipulations. Growth will be monitored in the presence of genetic libraries, chemicals, drugs, etc. to identify those that give a selective growth advantage. Mutants and chemicals from a variety of sources will be tested.

In particular embodiments, methods of the present disclosure relate to determining PLD inhibition caused by aS. This can be done, for example, using an assay utilizing temperature sensitive (ts) mutants of PLD to investigate the effect of aS expression on PLD activity in yeast. For example, a simple genetic screen (see, e.g., Xie Z, et al., 1998. Proc Natl Acad Sci USA 95:12346-52) allows modulators of this activity to be readily identified. Optionally, chemical modifiers of aS/PLD interaction can be identified by the same method.

In particular embodiments, methods of the present disclosure relate to determining proteasomal impairment caused by aS. This can be done, for example, be means of an assay that utilizes fusions between ubiquitin and β-galactosidase molecules with different N termini (see, e.g., Bachmair A, et al., 1986. Science. 234:179-86). Such assay allows qualitative assessment of proteasome activity in yeast. Using this assay system, it was determined that high levels of wildtype aS-GFP which forms inclusions in the yeast cytosol, reduce proteasome activity relative to cells expressing equivalent levels of GFP alone. This assay makes it possible to quantify and compare the behavior of distinct aS forms (wildtype vs. mutants) as well as to determine the effect of chaperones, other aS-interacting proteins, and conditions such as oxidative stress on the protein degradation arm of the quality control system. Optionally, pulse-chase experiments can be performed to measure half-lives and to assess proteasomal impairment more quantitatively.

In particular embodiments, methods of the present disclosure relate to determining oxidative stress caused by aS. Mitochondrial dysfunction and oxidative stress are clearly linked to diseases (e.g., Parkinson's disease) but in ways still poorly understood. Oxidative stress levels are readily manipulated in yeast by: 1) carbon sources that enhance or repress respiratory mechanisms; 2) mutations in components that regulate the redox state of cells and their response to changes in that state; and 3) mutations and/or drugs that affect mitochondria and the production of reactive oxygen species (ROS). Based on the experimental results described herein, it is clear that aS sensitization is highly specific. Mitochondrial poisons, metals, and chaperone mutations that sensitize cells to oxidative stress with huntingtin protein show only partial overlap with those that sensitize the cell to aS. For example, ROS levels in yeast cells can be measured by examining the conversion of hydroethidium to ethidium by superoxide radical. Cells expressing aS were found to have increased levels of superoxide radical production when compared to cells not expressing aS. The effect of growth conditions, mutations, chemicals, and metals that influence the growth rate of aS-expressing yeast cells can be assessed by using methods well known in the art. Optionally, the levels of anti-oxidant defenses in cells expressing aS can be measured to investigate the connection between aS expression, oxidative stress, and oxidative defense.

In particular embodiments, methods of the present disclosure relate to genomics studies of aS expression in yeast cells. For example, whole-genome expression profiling can be performed with yeast, generating DNA microarrays specific for the predicted ˜6300 open reading frames (ORFs) present in the yeast genome. 70-mer oligos were used rather than PCR products, a Gene Machines printing robot and an Axon scanner. Microarray analysis can make it possible to relate gene expression changes to the biological effects of aS in yeast (and in mammalian cells). Alternatively, genes that are differentially expressed in aS expressing yeast cells relative to aS non-expression can be identified by other well known methods such as differential PCR display, or subtractive hybridization.

Certain embodiments provide methods of further testing those potential drugs that have been identified in the yeast system, in other model systems. The model systems include, but are not limited to, worms, flies, mammalian cells, and in vivo animal models (e.g., an aS transgenic mouse).

### Methods of Treatment

Certain aspects of the present disclosure relate to methods of treating a subject suffering from an aS associated disease and/or a protein misfolding disease. As described above, aS associated diseases include, but are not limited to Parkinson's Disease, Parkinson's Disease with accompanying dementia, Lewy body dementia, Alzheimer's disease with Parkinsonism, and multiple system atrophy. Protein misfolding diseases include many neurodegenerative diseases (e.g., Parkinson's disease, Alzheimer's disease, Huntington's disease, and prion diseases) and non-neuronal diseases (e.g., type 2 diabetes).

Certain embodiments contemplate initial testing and treatment of animal-models with candidate drugs identified by screens described herein. Suitable animal-model for the aS associated diseases and/or protein misfolding diseases will be selected, and treatment will involve the administration of the drugs, in an appropriate pharmaceutical formulation, to the animal. Administration will be by any route that could be utilized for clinical or non-clinical purposes, including but not limited to oral, nasal, buccal, or topical. Alternatively, administration may be by intratracheal instillation, bronchial instillation, intradermal, subcutaneous, intramuscular, intraperitoneal or intravenous injection. Specifically contemplated routes are systemic intravenous injection, regional administration via blood or lymph supply, or directly to an affected site. Determining the effectiveness of a compound in vivo may involve a variety of different criteria.

In certain embodiments, the present disclosure provides methods of treating a subject (patient or individual) suffering from an aS associated disease and/or a protein misfolding disease. In other embodiments, the disclosure provides methods of preventing or reducing the onset of such diseases in a subject. For example, an individual who is at risk of developing Parkinson's disease (e.g., an individual whose family history includes Parkinson's disease) and/or has signs he/she will develop Parkinson's disease can be treated by the present methods. These methods comprise administering to the individual an effective amount of a compound that are identified by the screening methods as described above. These methods are particularly aimed at therapeutic and prophylactic treatments of animals, and more particularly, humans.

### Formulation and Administration

In certain embodiments, candidate drugs (compounds) may be formulated in combination with a suitable pharmaceutical carrier. Such formulations comprise a therapeutically effective amount of the drug, and a pharmaceutically acceptable carrier (excipient). Examples of suitable carriers are well known in the art. To illustrate, the pharmaceutically acceptable carrier can be an aqueous solution or physiologically acceptable buffer. Optionally, the aqueous solution is an acid buffered solution. Such acid buffered solution may comprise hydrochloric, sulfuric, tartaric, phosphoric, ascorbic, citric, fumaric, maleic, or acetic acid. Alternatively, such carriers include, but are not limited to, saline, buffered saline, dextrose, water, glycerol, ethanol, and combinations thereof. Formulations will suit the mode of administration, and are well within the skill of the art.

In certain embodiments of such methods, one or more drugs can be administered, together (simultaneously) or at different times (sequentially). In addition, such drugs can be administered with another type(s) of drug(s) for treating a protein misfolding disease. For example, the identified drug may be administered together with Levodopa (L-DOPA) for treating Parkinson's disease.

The phrase “therapeutically effective amount,” as used herein, refers to an amount that is sufficient or effective to prevent or treat (prevent the progression of or reverse) a protein misfolding disease, including alleviating symptoms of such diseases.

The dosage range depends on the choice of the drug, the route of administration, the nature of the formulation, the nature of the subject's condition, and the judgment of the attending practitioner. Wide variations in the needed dosage, however, are to be expected in view of the variety of drugs available and the differing efficiencies of various routes of administration. For example, oral administration would be expected to require higher dosages than administration by intravenous injection. Variations in these dosage levels can be adjusted using standard empirical routines for optimization, as is well understood in the art.

The following are examples of the practice of the invention. They are not to be construed as limiting the scope of the invention in any way.

### EXAMPLES

### Materials and Methods

The following materials and methods were used in work described herein.

A) Plasmid Constructions

Alpha-synuclein cDNA was a kind gift from Dr. Peter Lansbury. WT, A53T or A30P sequences were cloned into p426GPD, p416GPD, p423GPD, p425GPD and p426GAL (Mumberg et al., Gene, 156:119-122) as SpeI-HindIII-digested products of PCR amplification (primers 5′GGACTAGTATGGAT GTATTCA TGAAAGG3′ and 5′GGGGAAGCTTT TATTAGGCTTCAGGTTCGTAGTC3′; SEQ ID NO:7). GFP, CFP and YFP fusions were constructed by inserting the XFP (X meaning G, C or Y) coding sequence in frame with alpha-synuclein in the same vectors. The XFP fusions, together with the GALL-10 promoter and CYC1 terminator were subcloned into the integrative plasmids pRS306 and pRS304 as SacI/KpnI fragments.

B) Yeast Strains and Genetic Procedures

The laboratory yeast strain W303 (Mat a can1-100, his3-11,15, leu2-3,112, trp1-1, ura3-1, ade2-1) was used for our studies. Yeast strains were grown and manipulated following standard procedures. Yeast transformations were carried out by the standard lithium acetate procedure (Ito et al., 1983).

Yeast cells bearing disruptions of genes known to play important roles in drug efflux and cell permeability (PDR1, PDR3, PDR5, and ERG6) were generated by targeting these genes using short-flanking homology PCR (SFH-PCR), with the kanMX4 cassette. Correct ORF replacements were verified by PCR.

The integrative plasmids pRS304 and pRS306 bearing the alpha-synuclein constructs were linearized, by restriction digestion within the auxotrophic marker regions, and transformed into yeast. Heterologous gene expression was verified by western blot and fluorescence microscopy.

C) Spotting Experiments

Yeast cells were routinely grown overnight at 30° C. or at room temperature in selective media until they reached log or mid-log phase. Cells were then counted using a hemocytometer and diluted to 1×10⁶ cells/ml. Five serial dilutions (five fold) were made and cells were spotted onto media containing chemicals/drugs to screen.

### Example 1

### Formation of Intracellular Inclusion and Amyloid Fibers

Alpha-synuclein has the property of forming intracellular inclusions in neurons and forming amyloid fibers in vitro. Deposition of insoluble fibril proteins in tissues is a characteristic of diseases associated with protein misfolding. Most common of these diseases are neurodegenerative diseases (e.g., Parkinson's disease, Alzheimer's disease, Huntington's disease, and prion diseases), and other diseases such as type 2 diabetes. Agents that can prevent protein aggregation and fibril formation are being actively sought. However, methods of identifying such agents are limited.

Both wildtype aS and the A53T and A30P mutants form amyloid fibers, but biochemical and cell biological properties of these proteins differ. In a purified system, the A53T mutant fibrilizes faster than the A30P mutant and wildtype aS protein, whereas the A30P mutant forms an oligomeric species faster (Conway K A, et al., 2000. Proc Natl Acad Sci USA. 97:571-6). In primary neurons, fusions of aS with the green fluorescent protein (GFP) show that the wildtype aS and the A53T mutant behave in a similar way, and are capable of forming inclusions. However, A30P does not form similar inclusions under these conditions McLean P J, et al., 2001. Neuroscience. 104:901-12). Alpha synuclein associates with several types of membranes and lipid vesicles rich in acidic phospholipids (McLean P J, et al., 2000. J Biol Chem 275:8812-6; Jo E, et al., 2000. J Biol Chem 275:34328-34; Perrin R J, et al., 2000. J Biol Chem. 275:34393-8). However, the A30P mutant binds less effectively to acidic phospholipids and is less prone to form alpha-helices in its lipid-bound conformation (Perrin R J, et al., 1999. Soc. Neurosci. 25:27.11). Finally, the interaction between synphilin-1 (another aS-interacting protein) and the A53T mutant is twice as strong as that of the wildtype aS or the A30P mutant (Engelender S, et al., 1999. Nat. Genet. 22:110-114).

Alpha synuclein is toxic in both human embryo kidney cells (HEK 293) and in the human neuroblastoma cell line SK-N-SH, in a dose-dependent manner. The A53T and A30P mutations increase cell toxicity; A53T increases toxicity more than A30P (Ostrerova N, et al., 1999. J. Neurosci. 19:5782-5791; Zhou W, et al., 1999. Soc. Neurosci. 25:27.15). Alpha synuclein is upregulated by serum deprivation in HEK 293 cells. Complementing this observation, alpha synuclein antisense constructs are cytoprotective under conditions of serum deprivation (Ostrerova N, et al., 1999. J. Neurosci. 19:5782-5791). Toxicity is variable in other cell types (Ko L, et al., 1999. Soc. Neurosci. 25:27.24; Hanin I, et al., 1999. Soc. Neurosci. 25:27.27).

A hallmark of PD is the selective vulnerability of dopaminergic neurons compared to other cell types expressing alpha synuclein. This may relate to the fact that dopaminergic neurons face unusually high oxidative stresses, in particular, from dopamine oxidation (Lotharius J, et al., 2000. J Biol Chem 275:38581-38588; Berman S B, et al., 1999. J Neurochem 73:1127-1137) and/or high iron levels (Double K L, et al., 2000. J Neural Transm Suppl 60:37-58). Some aspects of dopaminergic neurons make them preferentially vulnerable to alpha synuclein-mediated toxicity, but it is clear that toxicity is not restricted to them, and that other cell types provide important, more experimentally tractable, systems to study. When alpha synuclein is expressed in yeast, respiratory metabolism and oxidative stress influence toxicity strongly.

### Example 2

### Inhibition of Phospholipase D (PLD)

Alpha synuclein was recently identified as a potent and selective inhibitor of mammalian phospholipase D2 (PLD2) (Jenco J M, et al., 1998. Biochemistry. 37:4901-9). Purified native PLD2 enzyme from mouse brain and recombinant PLD2 was employed in reconstitution assays to identify modulators of PLD activity. A mixture of alpha synuclein and beta-synuclein (bS) was discovered to be a potent inhibitor of PLD2 activity but not of PLD1. Providing a possible functional context, PLD activation is directly involved in membrane trafficking (Ktistakis N T, et al., 1996. J Cell Biol. 134:295-306) and cytoskeletal reorganization (Cross M J, et al., 1996. Gun Biol. 6:588-97). Specifically, PLD is thought to function in regulating vesicular movement either by activating a downstream effector essential for trafficking and/or by altering the local structural characteristics of membranes (Pertile P, et al., 1995. J Biol Chem. 270:5130-5). Moreover, there appears to be a direct requirement for the production of phosphatidic acid (PA) by PLD in the in vitro formation of coated vesicles from mammalian Golgi cisternae (Ktistakis N T, et al., 1996. J Cell Biol. 134:295-306). Thus, alpha synuclein might affect vesicle trafficking in part by influencing PLD functioning.

### Example 3

### Studies of Membrane Association

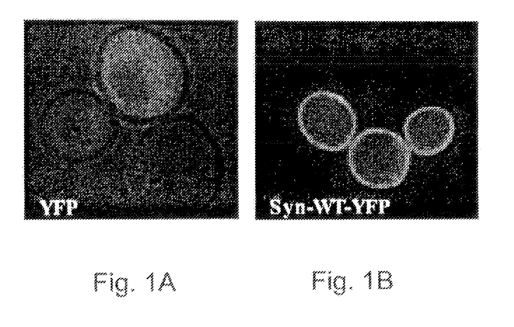

When the WT and A53T proteins are expressed at a low level, clear association membrane association is observed. The experiment of FIGS. 1A-1B employed fusions of aS with YFP (yellow FP). Similar results were obtained with GFP, and CFP (Cyan FP) fusion. This suggests that even though a yeast cell differs from a human neuron in several major ways, it still provides the necessary environment for aS to localize in a normal manner.

### Example 4

### Aggregation of aS in the Yeast Cytoplasm

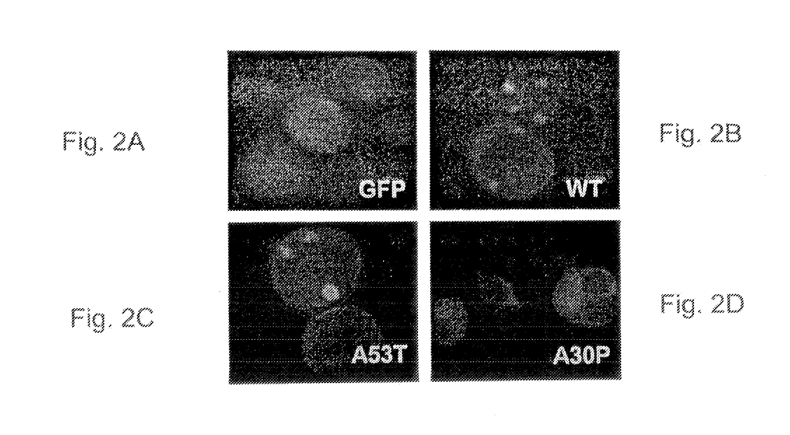

Expressing aS-GFP fusions at higher levels in yeast cytoplasm results in the formation of inclusions by WT aS and the A53T mutant, but not by GFP alone nor by A30P (FIGS. 2A-2D). Similar results have been reported in primary neuron cultures expressing similar fusion proteins (McLean P J, et al., 2001. Neuroscience. 104:901-12), supporting the idea that the behavior of aS in the yeast environment resembles that of mammalian cells in many aspects.

### Example 5

### Ubiquitination of aS

To investigate whether aS inclusions are ubiquitinated, as is the case with inclusions in other protein misfolding and mistrafficking diseases, cells were co-stained with anti-ubiquitin antibodies. Some, but not all, of the inclusions are ubiquitin-positive. Cells that contain ubiquitin-positive inclusions also showed reduced turnover of a protein reporter for proteasome activity.

### Example 6

### Toxicity of aS Expression in Yeast

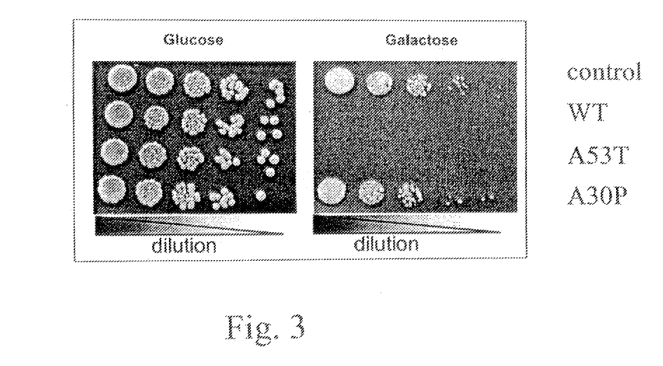

Several groups have shown that high levels of WT and A53T aS are toxic to mammalian cells (Ostrerova N, et al., 1999. J. Neurosci. 19:5782-5791; Zhou W, et al., 1999. Soc. Neurosci. 25:27.15). Results of work presented herein show that WT aS and A53T, but not A30P, is toxic in yeast (FIG. 3). Cells expressing aS alone, or aS-GFP, -YFP and -CFP fusions behave identically. Toxicity is dosage dependent. Cells that contain one integrated copy of an aS-GFP fusion gene under the regulation of a galactose-inducible promoter showed moderate growth defects; cells with two copies have extreme defects (FIG. 3). Under conditions that repress expression (growth in glucose) there is no growth difference between strains carrying these constructs.

High levels of toxicity, with two integrated genes, provide the best mechanism for finding factors that reduce toxicity. Low levels of toxicity, with one copy, provide a more sensitive system for testing factors that modulate toxicity. Using the single copy, low expression strains we observed that cells expressing aS were far more susceptible oxidative stress, mitochondrial poisons, and iron than control cells.

### Example 7

### Interactions of aS with htt

Previous investigations of polyglutamine (PQ) aggregates by other researchers revealed that in addition to ubiquitin these were often reactive for aS (Charles V, et al., 2000. Neurosci Lett. 289:29-32). Other reports described co-localization of htt and aS inclusions in mammalian cell lines (Waelter S, et al., 2001. Mol Biol Cell. 12:1393-407; Furlong R A, et al., 2000. Biochem J. 346 Pt 3:577-81). But it has not been clear if this was an idiosyncrasy of particular cell types, a trivial general aggregation problem, or a conserved property of interaction between the two proteins.

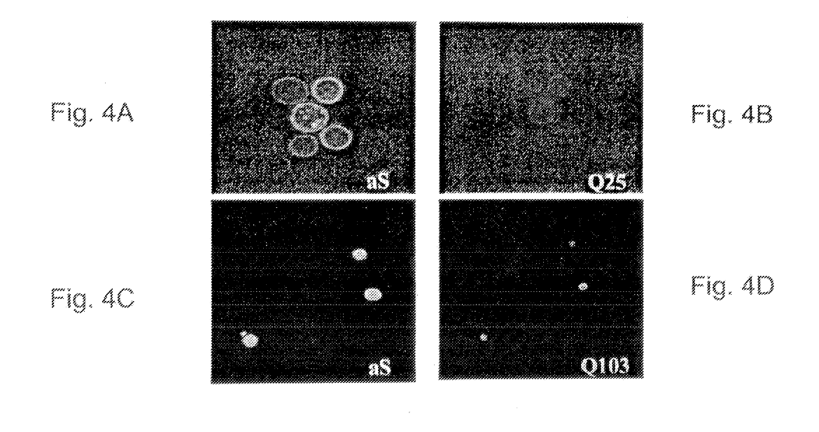

Yeast cells were engineered to express aS-YFP fusions together with htt exon 1-CFP fusion with normal and mutant lengths of PQ stretches. In cells expressing expanded stretches of PQ, such as 72 and 103 PQs, the localization of aS was altered and a substantial fraction of cells showed co-localization (FIGS. 4A-4D).

Co-aggregation of aS and htt was highly specific. Co-localization was not observed of aS with aggregates produced by GFP fusions of the mammalian prion protein (PrP), transthyretin (TTR), or other aggregation-prone proteins in the yeast cytoplasm.

### Example 8

### PLD Inhibition by aS

As stated above, aS was found to inhibit PLD2 activity in a reconstituted system. To investigate whether inhibition of PLD activity was a general property of aS, a search was carried out for the yeast PLD homologue that might be closest to the human PLD2. The yeast SPO14 gene encodes a PLD with higher homology to human PLD2 than to human PLD1 (Charles V, et al., 2000. Neurosci Lett. 289:29-32). Remarkably, using a “sec 14 bypass assay” (Xie Z, et al., 1998. Proc Natl Acad Sci USA 95:12346-52), it was observed that aS inhibits Spo14p function in yeast. Thus the ability of aS to inhibit PLD2 function is a highly conserved property that is likely to be intimately related to aS function in eukaryotic cells.

### Other Embodiments

It is to be understood that, while the invention has been described in conjunction with the detailed description thereof, the foregoing description is intended to illustrate and not limit the scope of the invention. Other aspects, advantages, and modifications of the invention are within the scope of the claims set forth below.

What is claimed is:

1. A yeast cell comprising an expression construct comprising a nucleic acid encoding a protein comprising an alpha synuclein, wherein the expression construct is integrated in the genome of the yeast cell, and wherein expression of the nucleic acid is regulated by an inducible promoter, such that induction of production of the protein is toxic to the yeast cell.

2. The yeast cell of claim 1, wherein the cell comprises two integrated copies of the expression construct.

3. The yeast cell of claim 1, wherein induction of expression of the nucleic acid renders the cell non-viable.

4. The yeast cell of claim 1, wherein induction of expression of the nucleic acid arrests growth of the cell.

5. The yeast cell of claim 1, wherein the alpha synuclein is human alpha synuclein.

6. The yeast cell of claim 1, wherein the alpha synuclein is a mutant alpha-synuclein.

7. The yeast cell of claim 1, wherein the yeast is Saccharomyces cerevisiae, Saccharomyces uvae, Saccharomyces kluyveri, Schizosaccharomyces pombe, Kluyveromyces lactis, Hansenula polymorpha, Pichia pastoris, Pichia methanolica, Pichia kluyveri, Yarrowia lipolytica, Candida sp., Candida utilis, Candida cacaoi, Geotrichum sp., or Geotrichum fermentans.

8. The yeast cell of claim 1, wherein the inducible promoter is GAL1-10, GAL1, GALL, GALS, GPD, ADH, TEF, CYC1, MRP7, MET25, TET, VP16, or VP16-ER.

9. The yeast cell of claim 1, wherein the expression construct is an integrative plasmid.

10. The yeast cell of claim 9, wherein the integrative plasmid is pRS303, pRS304, pRS305, or pRS306.

11. The yeast cell of claim 1, wherein the protein is a fusion protein comprising a detectable protein.

12. The yeast cell of claim 11, wherein the detectable protein is a fluorescent protein, an enzyme, or an epitope.

13. The yeast cell of claim 12, wherein the detectable protein is a fluorescent protein selected from the group consisting of a red fluorescent protein, green fluorescent protein, blue fluorescent protein, yellow fluorescent protein, and cyan fluorescent protein.

14. The yeast cell of claim 1, wherein at least one gene that encodes a polypeptide involved in drug efflux or cell permeability is disrupted.

15. The yeast cell of claim 14, wherein the at least one gene is PDR1, PDR3, or ERG6.

16. The yeast cell of claim 14, wherein the at least one gene is PDR5.

17. A yeast cell expressing a toxicity-inducing amount of a protein comprising an alpha synuclein.

18. The yeast cell of claim 17, wherein the cell comprises an integrated expression construct comprising a nucleic acid encoding the protein.

19. The yeast cell of claim 17, wherein the cell comprises two integrated copies of an expression construct comprising a nucleic acid encoding the protein.

20. The yeast cell of claim 17, wherein the alpha synuclein is human alpha synuclein.

21. The yeast cell of claim 17, wherein the alpha synuclein is a mutant alpha-synuclein.

22. The yeast cell of claim 17, wherein the yeast is Saccharomyces cerevisiae, Saccharomyces uvae, Saccharomyces kluyveri, Schizosaccharomyces pombe, Kluyveromyces lactis, Hansenula polymorpha, Pichia pastoris, Pichia methanolica, Pichia kluyveri, Yarrowia lipolytica, Candida sp., Candida utilis, Candida cacaoi, Geotrichum sp., or Geotrichum fermentans.

23. A method of identifying a compound that prevents or suppresses alpha-synuclein-induced toxicity, the method comprising:
culturing the yeast cell of claim 1 in the presence of a candidate agent and under conditions that allow for expression of the protein at a level that, in the absence of the candidate agent, is sufficient to induce toxicity in the yeast cell; and
determining whether toxicity in the yeast cell is less in the presence of the candidate agent as compared to in the absence of the candidate agent,
wherein if the toxicity is less in the presence of the candidate agent, then the candidate agent is identified as a compound that prevents or suppresses alpha-synuclein-induced toxicity.

24. The method of claim 23, wherein the alpha synuclein is human alpha synuclein.

25. The method of claim 23, wherein the alpha synuclein is a mutant alpha-synuclein.

26. The method of claim 25, wherein the mutant alpha-synuclein is mutant human alpha-synuclein A53T.

27. A method of identifying an extragenic suppressor of alpha-synuclein-induced toxicity, the method comprising:
culturing the yeast cell of claim 1, wherein an endogenous gene of the yeast cell has been disrupted, under conditions that allow for expression of the protein at a level that, in the absence of the disruption of the endogenous gene, is sufficient to induce toxicity in the yeast cell; and
determining whether toxicity in the yeast cell is less in the presence of the disruption of the endogenous gene as compared to in the absence of the disruption of the endogenous gene,
wherein if the toxicity is less in the presence of the disruption of the endogenous gene, then the disrupted endogenous gene is identified as an extragenic suppressor of alpha-synuclein-induced toxicity.

28. A method of identifying a compound that modulates alpha-synuclein localization to a plasma membrane, the method comprising:
culturing, in the presence of a candidate agent, a yeast cell ectopically expressing a protein comprising an alpha-synuclein; and
determining whether localization of the protein to the plasma membrane in the yeast cell is altered in the presence of the candidate agent as compared to in the absence of the candidate agent,
wherein if localization of the protein to the plasma membrane is altered in the presence of the candidate agent, then the candidate agent is identified as a compound that modulates alpha-synuclein localization to the plasma membrane.

29. A method of identifying a compound that inhibits the aggregation or formation of inclusions of alpha-synuclein, the method comprising:
culturing, in the presence of a candidate agent, a yeast cell ectopically expressing a protein comprising an alpha-synuclein; and
determining whether cytoplasmic aggregation or inclusion formation of the protein is less in the presence of the candidate agent as compared to in the absence of the candidate agent,
wherein if aggregation or formation of inclusions of the protein is less in the presence of the candidate agent, then the candidate agent is identified as a compound that inhibits the aggregation or formation of inclusions of alpha-synuclein.

30. A method of identifying a compound that promotes disaggregation of alpha-synuclein, the method comprising:
providing a yeast cell ectopically expressing a protein comprising an alpha-synuclein, wherein the cell comprises cytoplasmic aggregates or inclusions of the protein;
contacting the yeast cell with a candidate agent; and
determining whether cytoplasmic aggregation or inclusion formation of the protein is reduced in the presence of the candidate agent as compared to in the absence of the candidate agent,
wherein if aggregation or formation of inclusions of the protein is reduced in the presence of the candidate agent, then the candidate agent is identified as a compound that promotes disaggregation of alpha-synuclein.

31. A method of identifying a compound that prevents or suppresses proteasomal impairment caused by alpha-synuclein, the method comprising:
culturing, in the presence of a candidate agent, a yeast cell ectopically expressing a protein comprising an alpha-synuclein; and
determining whether proteasomal impairment in the cell is less in the presence of the candidate agent as compared to in the absence of the candidate agent,
wherein if proteasomal impairment in the cell is less in the presence of the candidate agent, then the candidate agent is identified as a compound that prevents or suppresses proteasomal impairment caused by alpha-synuclein.

32. A method of identifying a compound that prevents or suppresses phospholipase D (PLD) inhibition caused by alpha-synuclein, the method comprising:
culturing, in the presence of a candidate agent, a yeast cell ectopically expressing a protein comprising an alpha-synuclein; and
determining whether PLD inhibition in the cell is less in the presence of the candidate agent as compared to in the absence of the candidate agent,
wherein if PLD inhibition in the cell is less in the presence of the candidate agent, then the candidate agent is identified as a compound that prevents or suppresses PLD inhibition caused by alpha-synuclein.

33. A method of identifying a compound that prevents or suppresses oxidative stress caused by alpha-synuclein, the method comprising:
culturing, in the presence of a candidate agent, a yeast cell ectopically expressing a protein comprising an alpha-synuclein; and
determining whether oxidative stress in the cell is less in the presence of the candidate agent as compared to in the absence of the candidate agent,
wherein if oxidative stress in the cell is less in the presence of the candidate agent, then the candidate agent is identified as a compound that prevents or suppresses oxidative stress caused by alpha-synuclein.

34. A method of identifying a compound that reduces or inhibits an interaction of alpha-synuclein with an alpha-synuclein associated protein, the method comprising:
culturing, in the presence of a candidate agent, a yeast cell ectopically expressing (i) a first protein comprising an alpha-synuclein, and (ii) a second protein comprising an alpha-synuclein associated protein; and
determining whether the interaction between the first protein and the second protein in the cell is less in the presence of the candidate agent as compared to in the absence of the candidate agent,
wherein if the interaction between the first protein and the second protein in the cell is less in the presence of the candidate agent, then the candidate agent is identified as a compound that reduces or inhibits the interaction of alpha-synuclein with the alpha-synuclein associated protein.

35. The method of claim 34, wherein the alpha-synuclein associated protein is dephospho-BAD, protein kinase C (PKC), mitogen-activated extracellular regulated kinase (ERK), synphilin-1, Huntington (htt), phospholipase D (PLD), or parkin.

36. The method of claim 34, wherein the alpha-synuclein associated protein is Tau.

37. A method of identifying an alpha-synuclein associated protein, the method comprising:
transforming a yeast cell with (i) a first expression construct encoding a first protein comprising an alpha-synuclein, and (ii) a second expression construct encoding a second protein comprising a candidate protein;
incubating the yeast cell under conditions that allow for expression of the first protein and the second protein; and
determining whether the first protein interacts with the second protein, wherein if an interaction occurs, the candidate protein is identified as an alpha-synuclein associated protein.

38. A method of identifying a gene that is involved in an alpha-synuclein associated disease, the method comprising:
isolating RNAs from a first yeast cell that ectopically expresses alpha-synuclein;
isolating RNAs from a second yeast cell that does not ectopically express alpha-synuclein;
comparing the RNAs isolated from the first yeast cell and the RNAs isolated from the second yeast cell; and
identifying an RNA that is present at a higher or lower level in the first cell relative to the second yeast cell, to thereby identify a corresponding gene that is involved in an alpha-synuclein associated disease.

39. The method of claim 38, wherein the RNA is identified by performing differential display.

40. The method of claim 38, wherein the RNA is identified by performing subtractive hybridization.

41. A method of treating an individual suffering from a protein misfolding disease, the method comprising administering to the individual a pharmaceutical composition comprising a therapeutically effective amount of a compound identified by the method of claim 23.

42. A method of treating an individual suffering from Parkinson's Disease, the method comprising administering to the individual a pharmaceutical composition comprising a therapeutically effective amount of a compound identified by the method of claim 23.


SEQUENCE LISTING (8 sequences)

SEQ ID NO: 1 - DNA, 423 bp, Homo sapiens
atggatgtattcatgaaaggactttcaaaggccaaggagggagttgtggctgctgctgagaaaaccaaacagggtgtggcagaagcagcaggaaagacaaaagagggtgttctctatgtaggctccaaaaccaaggagggagtggtgcatggtgtggcaacagtggctgagaagaccaaagagcaagtgacaaatgttggaggagcagtggtgacgggtgtgacagcagtagcccagaagacagtggagggagcagggagcattgcagcagccactggctttgtcaaaaaggaccagttgggcaagaatgaagaaggagccccacaggaaggaattctggaagatatgcctgtggatcctgacaatgaggcttatgaaatgccttctgaggaagggtatcaagactacgaacctgaagcctaa

SEQ ID NO: 2 - protein, 140 aa, Homo sapiens
MDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKNEEGAPQEGILEDMPVDPDNEAYEMPSEEGYQDYEPEA

SEQ ID NO: 3 - DNA, 405 bp, Homo sapiens
atggacgtgttcatgaagggcctgtccatggccaaggagggcgttgtggcagccgcggagaaaaccaagcagggggtcaccgaggcggcggagaagaccaaggagggcgtcctctacgtcggaagcaagacccgagaaggtgtggtacaaggtgtggcttcagtggctgaaaaaaccaaggaacaggcctcacatctgggaggagctgtgttctctggggcagggaacatcgcagcagccacaggactggtgaagagggaggaattccctactgatctgaagccagaggaagtggcccaggaagctgctgaagaaccactgattgagcccctgatggagccagaaggggagagttatgaggacccaccccaggaggaatatcaggagtatgagccagaggcgtag

SEQ ID NO: 4 - protein, 134 aa, Homo sapiens
MDVFMKGLSMAKEGVVAAAEKTKQGVTEAAEKTKEGVLYVGSKTREGVVQGVASVAEKTKEQASHLGGAVFSGAGNIAAATGLVKREEFPTDLKPEEVAQEAAEEPLIEPLMEPEGESYEDPPQEEYQEYEPEA

SEQ ID NO: 5 - DNA, 384 bp, Homo sapiens
atggatgtcttcaagaagggcttctccatcgccaaggagggcgtggtgggtgcggtggaaaagaccaagcagggggtgacggaagcagctgagaagaccaaggagggggtcatgtatgtgggagccaagaccaaggagaatgttgtacagagcgtgacctcagtggccgagaagaccaaggagcaggccaacgcggtgagcgaggctgtggtgagcagcgtcaacactgtggccaccaagaccgtggaggaggcggagaacatcgcggtcacctccggggtggtgcgcaaggaggacttgaggccatctgccccccaacaggagggtgtggcatccaaagagaaagaggaagtggcagaggaggcccagagtgggggagactag

SEQ ID NO: 6 - protein, 127 aa, Homo sapiens
MDVFKKGFSIAKEGVVGAVEKTKQGVTEAAEKTKEGVMYVGAKTKENVVQSVTSVAEKTKEQANAVSEAVVSSVNTVATKTVEEAENIAVTSGVVRKEDLRPSAPQQEGVASKEKEEVAEEAQSGGD

SEQ ID NO: 7 - DNA, 28 bp, Artificial Sequence
  primer
ggactagtatggatgtattcatgaaagg

SEQ ID NO: 8 - DNA, 34 bp, Artificial Sequence
  primer
ggggaagcttttattaggcttcaggttcgtagtc